(19) United States
(12) Patent Application Publication
(10) Pub. No.: US 2024/0325211 A1
(43) Pub. Date: Oct. 3, 2024

(54) DISPOSABLE HYGIENIC ARTICLE WITH MEANS FOR DIAGNOSTIC TESTING

(71) Applicant: Medline Industries, LP, Northfield, IL (US)

(72) Inventor: Christopher Nelson, Hiram, GA (US)

(21) Appl. No.: 18/740,454

(22) Filed: Jun. 11, 2024

Related U.S. Application Data
(63) Continuation of application No. 17/852,155, filed on Jun. 28, 2022, now Pat. No. 12,023,228.
Application No. 17/852,155 is a continuation of application No. 16/810,570, filed on Mar. 5, 2020, now Pat. No. 11,376,166.
Application No. 16/810,570 is a continuation of application No. 15/295,062, filed on Oct. 17, 2016, now Pat. No. 10,583,047.
Application No. 15/295,062 is a division of application No. 14/097,955, filed on Dec. 5, 2013, now Pat. No. 9,486,368.

(51) Int. Cl. A61F 13/15; A61B 5/00; A61B 5/145; A61B 10/00; A61F 13/42; A61F 13/45; A61F 13/49; A61F 13/514; A61F 13/84

(52) U.S. Cl. CPC A61F 13/15739; A61B 5/14507; A61B 5/6808; A61B 10/007; A61F 13/15585; A61F 13/15699; A61F 13/15723; A61F 13/15747; A61F 13/15756; A61F 13/49061; A61F 13/51498; A61F 13/84; A61B 2010/0003; A61B 2562/0295; A61F 13/42; A61F 2013/4587; A61F 2013/4593; A61F 2013/51468; A61F 2013/8473; Y10T 156/1062

(57) ABSTRACT

A disposable, wearable article can be used for collecting a sample and performing urinalysis. The article incorporates a test port feature to simplify this function. The test port has a resealable cover that can be used to selectively expose an aperture through the backsheet of the disposable article. The test port is used to position test devices, such as urine test strips to urine collected in the disposable article.

### CROSS-REFERENCE TO RELATED APPLICATIONS

This application is a continuation of U.S. patent application Ser. No. 17/852,155 filed on Jun. 28, 2022, which is a continuation of U.S. patent application Ser. No. 16/810,570 filed on Mar. 5, 2020 and issued as U.S. Pat. No. 11,376,166, which is a continuation of U.S. patent application Ser. No. 15/295,062 filed on Oct. 17, 2016 and issued as U.S. Pat. No. 10,583,047, which is a divisional of U.S. patent application Ser. No. 14/097,955 filed Dec. 5, 2013, issued as U.S. Pat. No. 9,486,368, and entitled “DISPOSABLE HYGIENIC ARTICLE WITH MEANS FOR DIAGNOSTIC TESTING.” Each application is incorporated herein by reference.

### FIELD OF THE INVENTION

The present invention relates generally to absorbent articles and, in particular, to the use of a disposable article for collecting a sample and performing urinalysis.

### BACKGROUND

Millions of people of all ages suffer from incontinence of the bowel or bladder. Whether an infant, adult, or elderly person, the underlying cause of incontinence varies but the method of treatment typically involves absorbent article products. Adult incontinent briefs, disposable diapers and underpads can alleviate some of the emotional and physical discomfort of incontinence by absorbing and containing liquid and other discharges from the human body to prevent body and clothing soiling.

Typical absorbent articles include a topsheet facing the wearer that permits fluid exudates to pass through and a backsheet that prevents the exudates from escaping from the absorbent article. Much advancement has been made in the art since the introduction of the disposable absorbent article, as shown, for example, in applicant's co-pending U.S. patent application Ser. No. 13/832,965, which is incorporated by reference herein. However, most of these articles are not adapted to aid the caregiver in the monitoring of the health of the wearer.

A problem encountered in post acute care settings is the increase in incidence of urinary tract infection (“UTI”). In addition, many patients in a managed care setting such as a nursing home require diagnostic testing. Many of these diagnostic tests require urinalysis, and many of these patients are also incontinent of urine. Therefore, collection of a urine specimen becomes a challenge for the caregivers in this environment. Current methods for collection of urine samples involve the extraction of urine from a used article. In the case of urine sampling from a baby, the sample is sometimes taken using a bag with an adhesive gasket, attaching to the wearer's skin. Finally, catheterization is sometimes used for sample collection. This practice can be attributed to an increase in incidence of UTI.

Certain attempts have been made in the art to include analysis of components of human waste, such as urine and feces, to provide indication of various specific health issues such as infections (e.g., urinary tract infections, etc.). For example, U.S. Pat. No. 5,468,236 issued to Everhart et al. on Nov. 21, 1995 discloses a disposable absorbent product that includes a chemically reactive means having an end point adapted to provide a visual indicator of the presence of a substance in mammalian bodily excrement. In another example, U.S. Pat. Nos. 7,365,238 and 8,217,217 issued to Diehl et al. on Apr. 29, 2008 and Jul. 10, 2012, respectively, disclose wearable articles with the capability to detect signals related to a dehydrated state of a wearer. However, the prior art fails to provide the caregiver with a convenient means of collecting samples and conducting a urinalysis.

Accordingly, a need exists for absorbent articles that provide a wearable article that can provide diagnostic functionality.

### BRIEF DESCRIPTION OF THE DRAWINGS

The foregoing and other advantages of the invention will become apparent upon reading the following detailed description and upon reference to the drawings.

FIG. 1 is a top plan view of an absorbent article in a substantially flat un-contracted position according to one embodiment of the invention.

FIG. 2 is an exploded perspective view of the absorbent article of FIG. 1, again in a substantially flat un-contracted position.

FIG. 3 is a perspective view of a second absorbent core of the absorbent article depicted in FIG. 1.

FIG. 4 is a perspective view of a first absorbent core of the absorbent article depicted in FIG. 1.

FIG. 5 is a perspective view of an absorbent article in accordance with an embodiment of the invention as viewed from an underside of the article.

FIG. 6 is a section view of a test port and cover in accordance with an embodiment of the invention.

FIG. 7 is a plan view of the patient facing side of a cover for a test port of an embodiment of the invention.

FIG. 8 is a cut-away side view of further embodiment of a test port cover with the cover in a closed position.

FIG. 9 illustrates the embodiment of FIG. 8 with the cover in an open position.

FIG. 10 is a cut-away side view of an absorbent article in accordance with the present invention.

FIGS. 11-16 are representative diagrams illustrating steps in an embodiment of a manufacturing process for manufacturing an absorbent article.

The drawings listed above are intended to convey to one of ordinary skill in the art the present invention and its embodiments. In some drawings certain elements have not been shown for clarity. While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention.

### DESCRIPTION OF ILLUSTRATIVE EMBODIMENTS

Absorbent articles as described herein generally include a moisture-pervious inner layer, an absorbent layer, and a moisture-impervious outer layer. Although the remainder of the description will be specifically directed to adult incontinence articles, such as disposable diapers, it is to be understood that the embodiments may also be implemented using other absorbent articles and that the properties and uses described below apply to these other absorbent articles as well. Throughout this application, the terms absorbent article and diaper are used interchangeably. However, it should be understood that the term diaper is intended to include other absorbent articles, such as training pants, incontinence pads, etc., as would be understood by one of ordinary skill in the art.

Embodiments of the invention are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, proximal and distal, and the like may be used solely to distinguish one entity or action from another entity or action without necessarily requiring or implying any actual such relationship or order between such entities or actions.

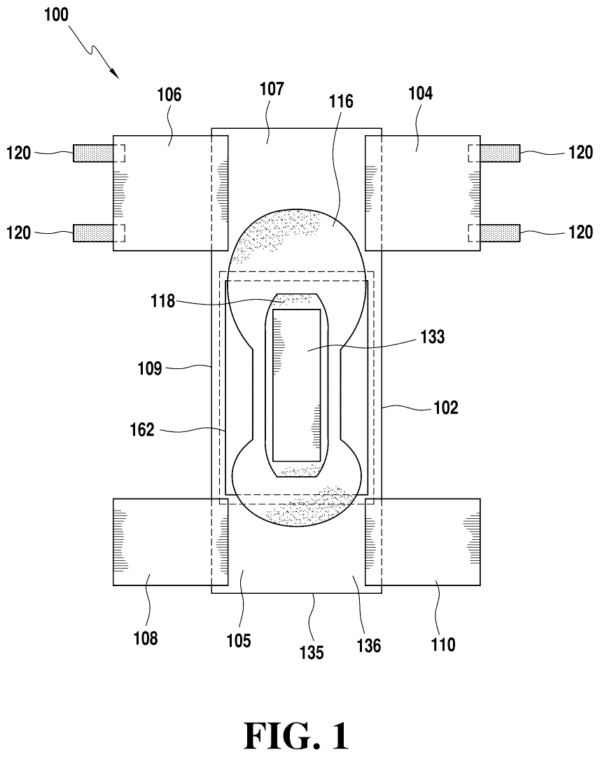

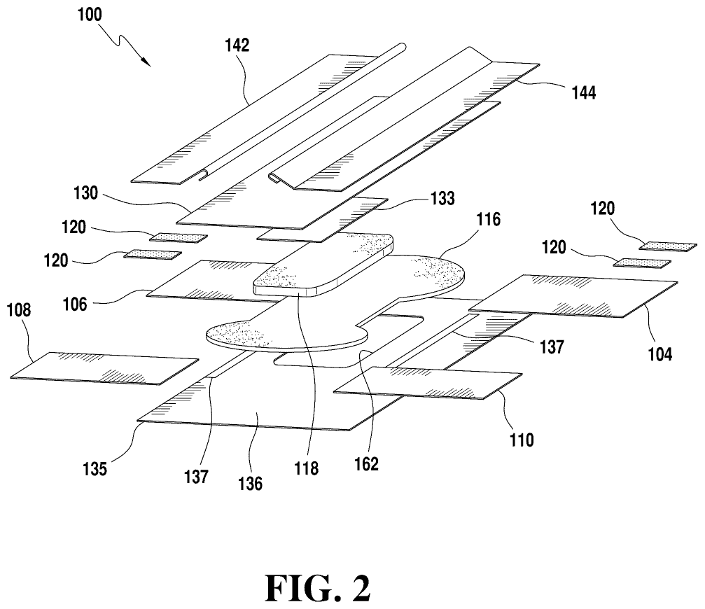

FIGS. 1 and 2 illustrate an exemplary non-limiting general embodiment of an absorbent article 100. FIG. 1 illustrates a plan view of the absorbent article 100 in a substantially flat un-contracted state. As shown in these figures, the absorbent article 100 generally consists of several layers, including an inner layer, an absorbent layer, and an outer layer. The inner layer faces a wearer and contacts the skin of the wearer when the absorbent article 100 is secured to the wearer. The inner layer may comprise a topsheet 130 that is composed of a moisture-pervious fabric suitable to allow bodily discharge to pass through the inner layer and be absorbed by the absorbent layer. Non-limiting examples of materials suitable to form the topsheet 130 include polypropylene, polyethylene, polyester, materials having hydrophobic properties, combinations thereof and/or the like. Additionally, the topsheet can be treated with a hydrophilic finish to improve pass through of liquids to diaper layers beneath the inner layer. Non-limiting examples of suitable hydrophilic finishes include stearic acid, melamine-based chemicals, fluorocarbon chemicals, and silicon based chemicals.

The plan view of FIG. 1 is shown from the top or patient contacting side of the absorbent article. The topsheet (130) and other components have been removed for clarity. FIG. 2 is an exploded perspective view of the absorbent article 100. Again, the article 100 is shown in a substantially flat un-contracted state with certain items removed for clarity.

As shown in FIG. 1, an embodiment of the absorbent article 100 comprises a chassis 102. The chassis 102 includes a front waist region 105, a back waist region 107, and a crotch region 109 that is disposed longitudinally between the front and back waist regions 105 and 107. The front waist region 105 and the back waist region 107 generally comprise those portions of the absorbent article 100 which, when worn, encircle the waist of the wearer. The crotch region 109 is that portion of the absorbent article 100 which, when the absorbent article 100 is worn, is generally positioned between the legs of the wearer.

The chassis 102 has a shape such that its outer perimeter is rectangular or at least substantially rectangular in the illustrative embodiment of the absorbent article 100. In other embodiment, there may be portions of the chassis that are shaped and/or removed, such as in the crotch region 109, for example, resulting in a narrower crotch region portion 109 to provide a contoured fit between the legs. Still other embodiments have different shaped chassis, such as hourglass shapes, T-shapes, and the like.

Rear side panels 104, 106 are coupled to and may extend from the back waist region 105. The disposable article may further include front side panels 108, 110 that are coupled to and may extend from the front waist region 105. The back region 107 is generally positioned against the back of the user. The front region 105 is generally positioned against the front of the user. The rear side panels 104, 106 are configured to wrap around a wearer's waist from back to front, extending from each side of the back waist region 105. The front side panels 108, 110 are configured to wrap around a wearer's waist from front to back. In this manner, rear side panel 106 can be connected to front side panel 108 and rear side panel 104 can be connected to front side panel 110 to couple the front region 105 to the back region 107. In this embodiment there are four side panels 104, 106, 108, 110. However, it should be recognized that other embodiments may be configured with more or fewer side panels. In particular, rear side panels 104, 106 may connect directly to an outside surface of front waist region 105 rather than to front side panels 108, 110.

The side panels may attach to the chassis 102 in a variety of manners as would be apparent to one of skill in the art. For example, as described in applicant's co-pending U.S. patent application Ser. No. 13/832,965. Alternatively, one or more of the side panels may be integrally formed, in whole or in part, with a backsheet 135 or topsheet 130 of the absorbent article. The backsheet 135 will have an outside surface 134 facing away from the patient wearing the absorbent article and an inside surface 136 facing toward the patient.

The rear side panels 104, 106 may also include fasteners 120. Fasteners 120 may comprise adhesive tape, hook and loop, snaps or any other appropriate fasteners as would be understood by one of ordinary skill in the art. As shown in the illustrative embodiment, rear side panel 104, 106 includes two fasteners 120. In a preferred embodiment, fasteners 120 can be configured to operatively couple rear side panels 104, 106 to a front region 105 of the diaper chassis 102. Alternative, fasteners 120 may also engage front side panels 108, 110 to attach rear side panels 104, 106, respectively. While FIG. 1 depicts rear side panels 104, 106 as including two fasteners 120, in some embodiments, more or fewer fasteners may be used. While FIG. 1 depicts fasteners 120 sized and shaped a particular way, in other embodiments, fasteners 120 can be a different size and/or shape. Alternatively, the front side panels 108, 110 may include fasteners in additions to, or in place of, the fasteners 120 attached to rear side panels 104, 106.

In another embodiment, the front region 105 and/or front panels 108, 110 may include added or modified features to reinforce or increase the affinity to the fastening device. Additionally, features may be employed to allow adhesive fasteners to be attached and detached from the fastening region multiple times. Those skilled in the art will recognize that there are multiple approaches to doing so via modification of the base material as well as additions of various materials. For example, fasteners 120 may incorporate the hook component of a hook-and-loop closure and portions of the front region 105 and/or front panels 108, 110 may be comprise a corresponding loop component. The surface of front region 105 and/or front panels 108, 110 may be treated to increase or decrease the affinity for the hook components. Alternatively, separate loop component material may be adhered to the surface of the front region 105 and/or front panels 108, 110.

Referring again FIGS. 1 and 2, embodiments of the absorbent article 100 include an absorbent layer. The absorbent layer may comprise an acquisition and/or distribution (“A/D”) layer 133, a first absorbent core 116, and a second absorbent core 118.

The liquid acquisition and/or distribution layers serves to rapidly acquire and then distribute acquired liquid to an absorbent core for retention. To achieve rapid acquisition and distribution, these layers often include cellulosic fibers. These layers can include cross-linked cellulosic fibers to impart bulk and resilience to the layer, and wood pulp fibers to increase the wicking of liquid within the layer and to facilitate distribution of the liquid throughout the layer and ultimately to another layer, such as a storage layer, that is in liquid communication with the distribution layer.

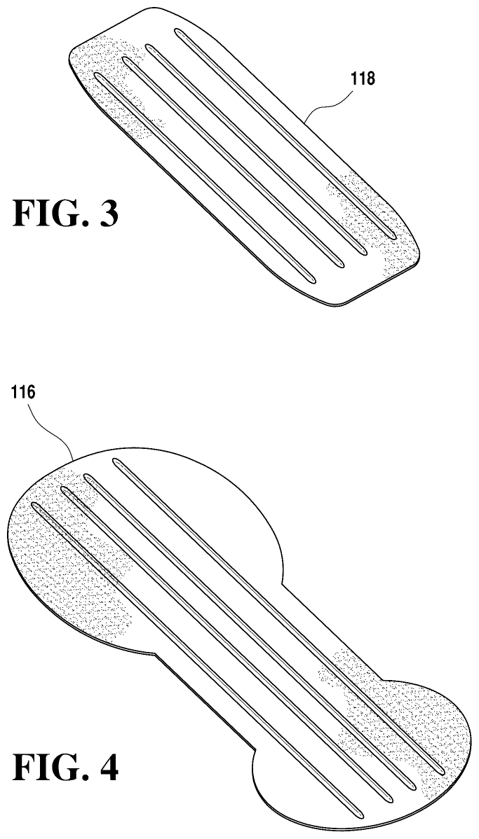

FIG. 3 is a perspective view of a top (facing towards wearer) of the second core 118, and FIG. 4 is a perspective view of a bottom side (facing away from a wearer) of the first core 116. Each of the first core 116 and second core 118 can be composed of similar material, and can be shaped depending on the size of the absorbent article, and whether it is intended for use by infants, children and/or adults. By way of example, and as shown in FIGS. 3 and 4, first core 116 can be larger and substantially hourglass shaped, whereas second core 118 can be smaller, relative to first core 116, and can be substantially rectangular shaped. In this manner, the absorbent article can include a large surface area of coverage provided by the first core 116, and the increased absorbency provided by the second core 118, without the additional bulk of a second core having the same size as the first core.

First core 116 is shown having an embossed bottom and second core 118 is shown having an embossed top. The embossed top of second core 118 and the embossed bottom of first core 116 provide increased longitudinal flow while reducing lateral flow, and, in this manner, reducing leakage. Said another way, the embossed top of second core 118 and the embossed bottom of first core 116 allows fluid to move longitudinally towards the front and the back of a wearer, as opposed to towards the legs of a wearer.

Each of the first core 116 and the second core 118 may be composed of any materials suitable for absorbing the fluids and discharge including, but not limited to, a fibrous material (e.g., fluffed wood pulp), a super absorbent polymer (“SAP”), or the combination of SAP and fibrous material. The SAP can be natural or synthetic and may be biodegradable. Non-limiting examples of SAP include polymers based on acrylate(s) such as sodium acrylate, potassium acrylate, and/or an alkyl acrylate(s) (e.g., methyl acrylate, ethyl acrylate, propyl acrylate, butyl acrylate, and hexyl acrylate). The absorbency of the diaper may vary depending upon whether it is intended for use by infants, children and/or adults.

While FIGS. 3 and 4 depict the first core 116 having an embossed bottom, and the second core 118 having an embossed top, in some embodiments, an absorbent article can have only a single core with no embossing, a single core with embossing on both, and/or other combinations of one or two cores each with embossing on one, both, or neither side. While the figures show absorbent articles include one or two cores, in some embodiments, absorbent articles can include more or fewer cores.

FIGS. 3 and 4 depict embossing as including four spaced apart embossing “lines,” in some embodiments. However, a core can include more or fewer embossing lines. In some embodiments, embossing lines can be adjacent one another, or can be a combination of adjacent and space apart embossing line. In this manner, the different combinations of embossing lines can define an embossing pattern. While FIGS. 3 and 4 depict embossing substantially along the entire width and length of each respective core, in some embodiments a core can have embossing substantially along an entire width and/or length, and/or a portion of a width and/or length.

In embodiments of the invention, the first 116 and second 118 cores may be created with or without the use of super absorbent polymer (SAP). While some literature indicates that the presence of SAP in a diaper used for urine testing is considered benign, manufacturing diapers without SAP for the benefit of accuracy is contemplated by the present invention and may be considered beneficial.

Returning to FIGS. 1 and 2, the absorbent article 100 may further include a set of leak guards and/or a set leg cuffs 142, 144, both known to those of ordinary skill in the art. Additionally, the exemplary absorbent article includes an outer layer or backsheet 135 and elastic bands 137. The elastic bands 137 can by used to gather the leg of the article around the user's leg, improving the fit of the absorbent article 100 and can improve the comfort of the wearer. Elastic bands and other elastic materials may be used at other places in the absorbent article in order to improve the fit and/or fluid retention of the article.

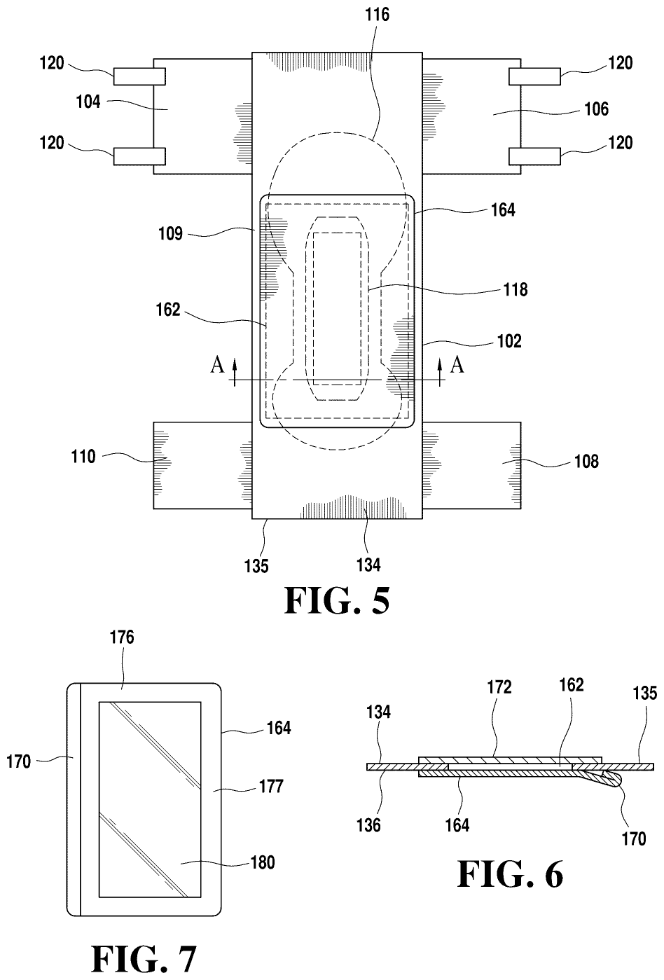

In further embodiments of the invention, the absorbent article includes a window or test port 162. The test port 162 passes through the backsheet 135 creating a through opening in the backsheet. As shown in FIG. 5, the test port 162 may have a cover 164 that surrounds the test port. The test port may be positioned in the crotch region 109 of the chassis 102 and may extend under at least a portion of the first absorbent core 116 and a portion of the second absorbent core 118. Alternatively, the test port may be position entirely or partially in the front 105 or back 107 waist regions.

As illustrated in FIG. 6, a section of the backsheet 135 is removed to form the test port 162. A cover 164 may be placed on the outside surface 134 of the backsheet surrounding the test port 162 so as to completely cover the test port. The cover may be releasably adhered to the backsheet.

The combination of the test port and cover allows the caregiver to collect and/or analyze a sample of urine that has collected in the absorbent article without the need to remove the article from the patient. Embodiments of the invention can be utilized with commercially available test strips, including but not limited to, for the identification of nitrites, ketones, proteins, pH level, hormone, Leukocytes, Bilirubin, Blood, Urobilinogen, and specific gravity. These indicators can aid in providing information about carbohydrate, metabolism (diabetes), kidney health, pregnancy, occult blood, acid/base balance, leukocytes (infection) and other health related conditions. In addition, embodiments of the invention may benefit the care facility by separating the storage conditions of the diaper from storage conditions of the test strip, thereby improving the shelf life and performance of the reagent strip.

Once inserted into the test port area 162 of the article 100, the reagent strip is utilized in the same manner as the strip would be when deployed in a traditional fashion (e.g., mid-stream sample collection into a cup).

The cover 164 may be of any appropriate, liquid impermeable material. For example, the cover may be formed of the same material as the backsheet 135. Alternatively, the cover 164 may be formed from a clear or translucent film or may comprise one or more clear portions. Use of a transparent material may allow the caregiver to place the test strip within the test port 162, return to the patient after the patient has voided, and read the strip (by comparison of the color chart to the reagent strip) without handling the strip or extracting urine from a diaper.

The cover 164 may comprise a finger lift 170 that aids the user in opening the cover 164. The finger lift 170 comprises a portion of the cover that is not adhered directly to the outside 134 of the backsheet 135. Additional embodiments may include a die cut or shaped tab. The caregiver can, accordingly, more easily grasp an edge of the cover 164 to peel the cover back from the backsheet, thereby opening the test port 162. The finger lift 170 may comprise a portion of the cover that is folded over upon itself to create a non-adhesive area at an edge of the cover.

In a further embodiment, the cover 164 may comprise a wetness indicating feature (not shown). The wetness indicator may include graphics, symbols, figures, writing or any other indicator comprised of color changing hotmelt adhesive, color changing ink, dissolving ink, or other means of indicating that the area has been exposed to an insult—as a result of urination—or that the area has been exposed to an insult for a sufficiently long duration of time for the reagent strip to function. In additional embodiments, graphics may be included in test port without the use of a wetness indicator.

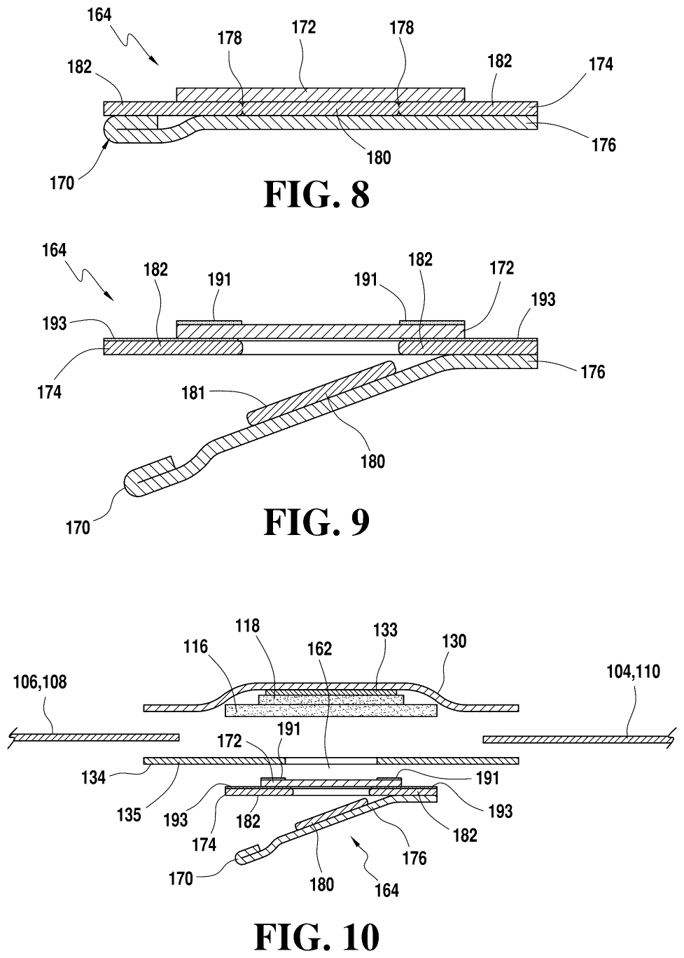

In addition, embodiments of the absorbent article may comprise a separation layer 172 that separates the absorbent cores 116, 118 from the cover 164. This separation layer 172 provides a barrier so that absorbent fluff is not exposed to the caregiver while inserting the test strip. The separation layer may be a nonwoven material. More particularly, the separation layer may be a hydrophilic nonwoven material. The separation layer may be positioned such that its edges overlap with the inside surface 136 of the backsheet 135, as shown in FIG. 6. Alternatively, the separation layer 172 may be positioned such that its edges overlap with the outside surface 134 of the backsheet 135, as shown in FIG. 10. In such an embodiment, an adhesive layer 191 may be applied in a window frame shape around the perimeter of the separation layer 172 in order to adhere the separation layer 172 to the backsheet 135. Additionally, the separation layer 172 may form part of the cover structure 164 as shown in FIG. 8.

As illustrated in FIGS. 7-9, embodiments of the cover 164 may comprise several components. FIGS. 7-9 illustrate a cross-section of the cover 164 generally along section line A-A of FIG. 5. Embodiments of the cover 164 include a separation layer 172 as described above. This separation layer 172 may be placed so that the edges overlap the outside surface 134 of the backsheet 135, surrounding the test port 162. The cover structure 164 may also include a base layer 174. The base layer 174 may overlap with both the separation layer and the backsheet outside surface 134 and be adhered to the backsheet 135 and/or separation layer 172 by a layer of adhesive 193 in a window frame shape around the perimeter of the base layer 174. In embodiments of the invention, the base layer 174 may comprise a bi-axially oriented polypropylene (“BOPP”) film. The cover 164 may further include an outer layer 176. The outer layer may cover, and be adhered to, out outer surface of the base layer 174. The outer layer may include a finger lift 170. Other films may also be used for the test port, including those used in packaging of food products, and may use low-tack, re162able adhesives.

The base layer 174 may include die cuts 178 extending through the base layer. In this manner, as the cover is pealed back using the finger lift 170, the base layer 174 separates into two parts, a central portion 180 that remains connected to the outer layer 176 and a peripheral portion 182 that remains attached to the backsheet 135. The base layer 174 may include a layer of film 181 applied to at least a portion of its top surface and extending at least over the central portion 180. This film, which may be a clear film, prevents the central portion of the base layer 174 from adhering to the separation layer 172. The caregiver may thereby access a test port within the absorbent article where a test strip can be placed.

The region 177 on an inside surface of the cover 164 surrounding the central portion 180, as illustrated in FIG. 7, may be an adhesive layer used to releaseably attach the outer layer 176 of the cover 164 of the test port 162 to the base layer 174. The base layer 174 is attached to the backsheet via a “picture frame” configuration of hotmelt adhesive 193 applied to a peripheral portion 182. Generally, this type of adhesive pattern is applied through the use of a hot melt application (slot coat) nozzle where the adhesive can be selectively turned on or off in prescribed regions across the width of the applicator.

Because the base layer 174 is only adhered to the backsheet in the outside perimeter 182, the center 180 of layer 174 may be torn away or deployed when peeling the outer layer 164 away as shown in FIG. 9. A die cut 178, which does not completely sever the layer, may be made through base layer 174 to allow the center portion 180 to separate with the cover 176 when the outer layer 176 is pulled away.

FIG. 10 illustrates an absorbent article having a test port according to embodiments of the invention. For clarity, certain elements that may be used in the article have not been illustrated in FIG. 10, for example leg cuffs 142, 144, among others. The exemplary diaper shown in FIG. 10 includes a topsheet 130. The topsheet is positioned above an A/D layer 133. The article may further include a first absorbent core 116 and a second absorbent core 118. A backsheet 135 having a test port 162 extending therethrough is positioned beneath at least a portion of the absorbent core 116. A separation layer 172 is positioned to cover the test port 162. The test port and separation layer are in turn covered by a cover 164 comprising a base layer 174, having a central portion 180 and a peripheral portion 182, and an outer layer 176. The outer layer 176 may include a finger lift 170. The diaper may also include side panels 104, 106, 108, 110 that attach to the topsheet 130 and/or the backsheet 135 and extend from the lateral edges of the chassis.

Manufacturing of embodiments of the present invention will include those steps employed in manufacturing an absorbent article as would be understood by one of ordinary skill in the art with the addition of those steps necessary to create a test port.

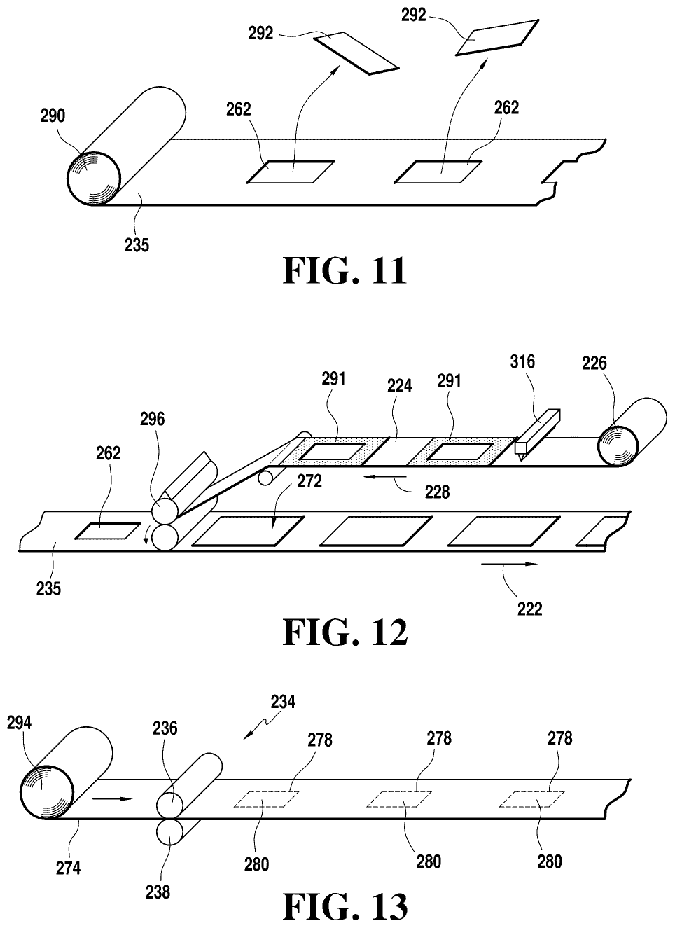

In a first process, as shown in FIG. 11, a film of backsheet material 235 is unrolled from a roll 290. A section of the film 292 is removed from the backsheet 235, leaving an aperture 262 in the film. The removed sections 292 are discarded or recycled as waste.

In a further step of the processes, as illustrated by FIG. 12, the backsheet 235 moves along a processing line in a direction shown by arrow 222. A web of permeable material 224 is unrolled from a second roll 226 in the direction show by arrow 228. The permeable material 224 may be a hydrophilic nonwoven material. An adhesive applicator 316 applies a window frame shaped adhesive pattern 291 to the permeable material 224. Sections of the nonwoven material are slip cut from the web of material 224 by a rotary cutter 296 and placed on the backsheet 235 to form nonwoven separation patches 272 such that the window frame adhesive 291 adheres the separation patches 272 around the periphery of apertures 262 in backsheet 235. Alternatively, permeable material 224 could be continuously applied in the same length as the backsheet 235 for simplification of the process.

In a second process, as illustrated by FIG. 13, a base layer 274 is unrolled from a roll of film material 294. A rotary die 234, including an anvil 236 and a pattern die 238, creates a perforated cut 278 through the base film 274, which creates a central portion 280 but does not completely sever the layer.

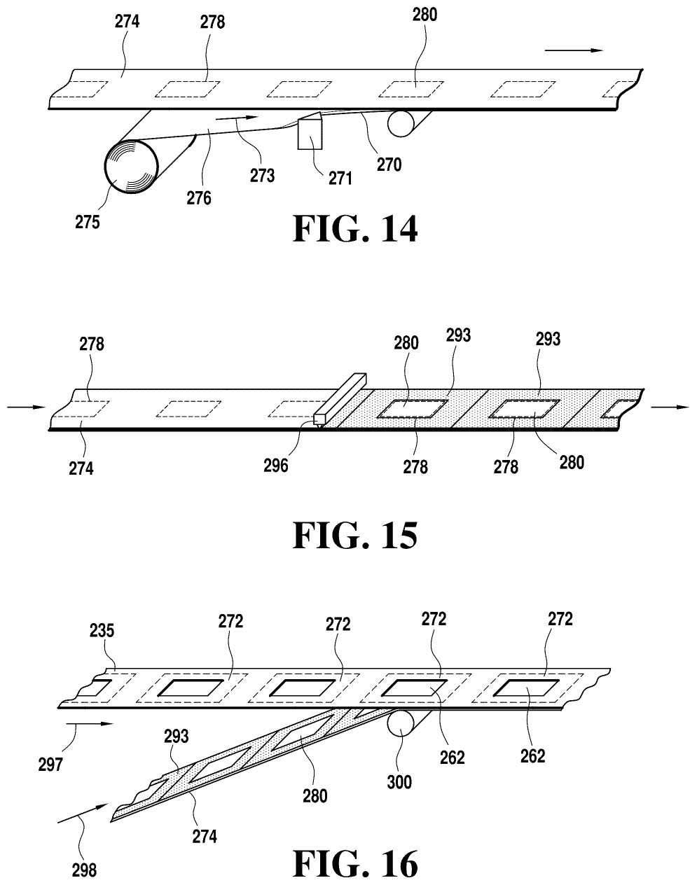

FIG. 14 illustrates a further step of the second process in which a web of an outer layer of material 276 is unwound from a roll 275 such that the outer layer is moving in a direction 273 while the base layer material 274 continues to move along the processing line. A fold 271 is applied to an edge of the outer layer 276 in order to create a finger lift fold 270. The outer layer of material 276 is adhered to an outer surface of the base layer 274.

FIG. 15 illustrates an additional step of the second process in which an adhesive applicator 296 applies a window frame shaped layer of adhesive 293 to the base layer 274 surrounding die cuts 278 and center portions 280.

As illustrated in FIG. 16, the backsheet 235 with separation patches 272 as produced by the first process, shown in FIGS. 11-12, moves in a direction illustrated by arrow 297. The base layer material 274 produced by the second process, shown in FIGS. 13-15, moves in a direction illustrated by arrow 298. The base layer material 274 moves through a slip/cut unit 300 that cuts the base layer material 274 and places it as discrete cover structures on an outer surface 234 of the backsheet 235 over the separation patches 272 and apertures 262 in the backsheet 235.

While the present invention has been described with reference to one or more particular embodiments, those skilled in the art will recognize that many changes may be made thereto without departing from the spirit and scope of the present invention. Furthermore, components from one embodiment can be used in other non-exclusive embodiments. Each of these embodiments and obvious variations thereof is contemplated as falling within the spirit and scope of the invention.

What is claimed is:

1. An absorbent article, comprising:
a front region;
a back region;
a crotch region;
an absorbent core;
a liquid impervious backsheet comprising an inside surface, an outside surface, and a test port extending through the backsheet from the outside surface to the inside surface to form an opening in the backsheet, the test port extending across at least a portion of the absorbent core; and
a cover positioned on the outside surface of the backsheet and selectively and resealably covering the backsheet test port, the cover comprising a clear portion.

2. The absorbent article of claim 1, wherein the test port is positioned in the crotch region.

3. The absorbent article of claim 1, wherein the test port is adapted to accommodate a test strip placed within the test port.

4. The absorbent article of claim 3, wherein the clear portion of the cover allows visual inspection of the test strip without opening the cover.

5. The absorbent article of claim 3, wherein the test strip is placed within the test port by opening the cover, inserting the test strip, and resealing the cover.

6. The absorbent article of claim 5, wherein the clear portion of the cover allows visual inspection of the test strip without reopening the cover after the cover has been resealed.

7. The absorbent article of claim 3, wherein the test port is not adapted to accommodate an absorbent core.

8. The absorbent article of claim 3, wherein the test strip is a reagent strip read by comparison of a color of the reagent strip to a color chart.

9. The absorbent article of claim 8, wherein the reagent strip can be read without handling the reagent strip after is has been placed in the test port.

10. The absorbent article of claim 1, wherein the absorbent core is positioned adjacent at least a portion of the backsheet inside surface.

11. The absorbent article of claim 1 further comprising a separation layer.

12. The absorbent article of claim 11, wherein the separation layer overlaps with at least a portion of the test port.

13. The absorbent article of claim 12, wherein the separation layer separates the absorbent core from the cover.

14. The absorbent article of claim 1, wherein the cover comprises a clear film.

15. The absorbent article of claim 1, wherein the cover comprises a translucent film.

16. The absorbent article of claim 1, wherein the cover comprises a transparent material.

17. An absorbent article, comprising:
a front region;
a back region;
a crotch region;
an absorbent core;
a liquid impervious backsheet comprising an inside surface, an outside surface, and a test port extending through the backsheet from the outside surface to the inside surface to form an opening in the backsheet, the test port extending across at least a portion of the absorbent core; and
a cover positioned on the outside surface of the backsheet and selectively and resealably covering the backsheet test port, the cover comprising a transparent portion.

18. The absorbent article of claim 17, wherein the test port is positioned in the crotch region.

19. An absorbent article, comprising:
a front region;
a back region;
a crotch region;
an absorbent core;
a liquid impervious backsheet comprising an inside surface, an outside surface, and a test port extending through the backsheet from the outside surface to the inside surface to form an opening in the backsheet, the test port extending across at least a portion of the absorbent core; and
a cover positioned on the outside surface of the backsheet and selectively and resealably covering the backsheet test port, the cover comprising a translucent portion.

20. The absorbent article of claim 19, wherein the test port is positioned in the crotch region.
